SEQUENCE LISTING (6 sequences)

SEQ ID NO: 1 - DNA, 26 bp, Rotavirus sp
cttcgccatggaggttctgtactcac

SEQ ID NO: 2 - DNA, 28 bp, Rotavirus sp
gtcgcgccatcggccgaattaattactc

SEQ ID NO: 3 - DNA, 26 bp, Astrovirus sp
aatcactccatgggaagctcctatgc

SEQ ID NO: 4 - DNA, 26 bp, Astrovirus
gtgacaagctcggccgcagatacagc

SEQ ID NO: 5 - DNA, 44 bp, Artificial Sequence
  Cloning vector plasmid pOM144 from Escherichia coli sp
aggaaaaaagcggccgccaccatggcgaaacgcgccagaccgtc

SEQ ID NO: 6 - DNA, 32 bp, Artificial Sequence
  Cloning vector plasmid pOM144 from Escherichia coli sp
aggaaaaaacggccgatgtttgcagggctagc

(19) United States
(12) Patent Application Publication
(10) Pub. No.: US 2010/0047763 A1
(43) Pub. Date: Feb. 25, 2010

(54) Plasmid Expression Vectors for Expression of Recombinant Rotavirus and Astrovirus Proteins or Epitopes

(76) Inventors: Ana Carolina Magalhäes Góes, Rio de Janeiro (BR); José Paulo Gagliardi Leite, Rio de Janeiro (BR); Márcia Tereyinha Baroni De Morales Souza, Rio de Janeiro (BR); Irene Trigueiros Araujo, Rio de Janeiro (BR); Jean Claude D'Halluin, Louvil (FR); Jose Godinho Da Silva, JR., Rio de Janeiro (BR)

Correspondence Address: MEREK, BLACKMON & VOORHEES, LLC, 673 S. WASHINGTON ST., ALEXANDRIA, VA, 22314, US

(21) Appl. No.: 12/083,910

(22) PCT Filed: Oct. 20, 2006
(86) PCT No.: PCT/BR2006/000222

§ 371 Date: Jul. 15, 2008

(30) Foreign Application Priority Data
Oct. 20, 2005 (BR) PI 0504441-3

(51) Int. Cl. C12Q 1/70; C12P 21/04; C07H 21/04; C12N 15/63

(52) U.S. Cl. 435/5; 435/69.6; 536/23.53; 435/320.1

(57) ABSTRACT

The present invention refers to the production of specific recombinant viral proteins intended for use in the construction of a diagnostic kit for the simultaneous detection of the two most important gastroenteric viruses, namely rotavirus and astrovirus.

### FIELD OF THE INVENTION

The present invention relates to the production of specific recombinant viral proteins in order to construct diagnostic kits for the identification of the two most important gastroenteric viruses, namely rotavirus and astrovirus.

### BACKGROUND OF THE INVENTION

Nowadays, viral gastroenteritis is one of the major public health problems worldwide due to its high rate of morbidity and mortality among children under 5 years of age. Although the mortality rate has decreased, the morbidity rate has remained stable over the last 4 decades. The amount of pathogens that causes gastroenteritis has increased in the last years due to distinct viral species that cause gastroenteritis and due to the discovery of several other agents that cause gastroenteritis.

Viruses have only been described as agents involved in the infantile gastroenteritis etiology in the last decades. Today, viruses, along with bacteria and parasites, are clearly recognized as pathogens involved in diarrheal diseases of medical importance. The most important viral agents that cause gastroenteritis are rotavirus, calicivirus, astrovirus and adenovirus.

Studies have shown that infection caused by rotavirus is the most common cause of diarrhea in children. The rate of hospitalized patients due to this pathology is approximately 35%. Globally, rotavirus has been considered the most important agent responsible for diarrhea in children under the age of 5.

The infection caused by rotavirus bands from mild (liquid diarrhea and limited duration) to severe (dehydration, fever and vomiting). However, some infections caused by rotavirus may be asymptomatic.

Rotavirus disease is uniformly distributed worldwide, but it has some distinct epidemiological characteristics in tempered climate areas and tropical climate areas.

The rotaviruses found in large amounts in infected children feces are transmitted through water, contaminated food and objects, personal contact and possibly transmitted through respiratory secretions. These mechanisms of transmission contribute to the high rate of dissemination of the rotavirus disease.

Studies have shown that since the discovery of astroviruses in 1975, these viruses have been considered the third major cause of viral gastroenteritis. The rate of detection of this virus may vary. The occurrence of astroviruses may be mainly due to nosocomial infections. Distinct from the rotavirus, astroviruses also occur in adolescents and adults. Studies have shown that astroviruses are found in 2, 4-17% of asymptomatic individuals. Many cases of astrovirus detection in clinical samples are associated with rotavirus positive samples.

Diarrhea caused by astrovirus occurs during a short period of time and has a lower severity. Other symptoms such as fever and vomiting occur less frequently. More severe symptoms occur in children or immunodeficient adults.

The traditional and universal hygiene practices such as hand washing, water and food quality control and adequate disposal of waste and sewage, which are indispensable for the prevention of diarrhea cases, have not been sufficient for the reduction of the incidence of infection caused mainly by rotavirus. This insufficiency is evidenced by the occurrence of cyclical epidemic disease in developed countries, which shows that the perspective of prevention of this disease will be achieved by means of the development of a vaccine capable of preventing the severe infections and, consequently, hospitalizations. However, so far, there is no available vaccine for this.

In Brazil, kits for identification of gastroenteritis etiological agents, particularly rotavirus, are commercialized. However, the culture of viral particles in specific cell cultures is necessary in order to produce raw material for these kits, which demands specialized people and optimization of processes.

To illustrate this fact, in patent document U.S. Pat. No. 5,298,244 the process of construction of a kit for the detection of etiologic agents that cause viral infections, particularly rotavirus, is described. The kit described in patent document U.S. Pat. No. 5,298,244 is based on the construction of viral particles derived from rotavirus proteins. These constructed particles consist of an inner capsid protein, VP6, combined with another protein or both proteins combined with other capsid proteins, such as VP4 and VP7. This construction can be used as a vaccine composition for the treatment and prevention of infections caused by rotavirus. The technique used in U.S. Pat. No. 5,298,244 is based on the recombinant eukaryotic expression using Autographa california-type cell cultures and a baculovirus genic expression system. Thus, as far as medium-scale production and maintenance are concerned, such technique demands a specific infrastructure that is costlier than the infrastructure necessary for the expression of recombinant proteins in the E. coli-type bacterial system, which is commonly used for most commercial recombinant proteins. U.S. Pat. No. 5,298,244 has no purification method in order to purify the complete particles formed in the system mentioned above. Consequently, it is not capable of generating purified particles for use in the induction and formation of specific antibodies in animal models and their subsequent utilization in diagnosis.

The recent discoveries of several etiologic agents that cause gastroenteritis highlight the astrovirus as an important pathogen. Therefore, there is a need for state-of-the-art development of new products targeting the diagnosis of the most important etiologic agents that cause gastroenteritis.

### SUMMARY OF THE INVENTION

The present invention relates to the production of specific recombinant viral proteins for application in the construction of diagnostic kits for the simultaneous detection of the two most important gastroenteric viruses, namely rotaviruses and astroviruses.

Thus, the first objective of the present invention is to characterize plasmid expression vectors containing specific epitope-coding regions of the rotavirus VP6 protein and astrovirus VP90 protein in E. coli system. For this purpose, molecular biology techniques, such as nucleotide sequencing and restriction profile analysis, are used.

Another objective of the present invention relates to the evaluation of both protein profile and yield of plasmid expression vectors-transformed clones grown in a small-scale bacterial culture. For this purpose, biochemistry techniques, such SDS-PAGE electrophoresis, are used.

Another objective of the present invention relates to the evaluation of the antigenicity of expressed epitopes by means of immunological techniques, such as Western-blot with commercial specific polyclonal antibodies and immunoenzymatic test.

Another objective of the present invention relates to the standardization of a purification method capable of generating highly purified recombinant epitopes of both rotavirus VP6 protein and astrovirus VP90 protein to be used as immunogens in animal models in the production of polyclonal antibodies.

Another objective of the present invention relates to the characterization of both polyclonal antibodies anti-rotavirus VP6 and anti-astrovirus VP90 derived from specific recombinant proteins. This characterization is performed by means of techniques such as Western-blot and immunoenzymatic tests.

### BRIEF DESCRIPTION OF THE DRAWINGS

FIG. 1 shows the schematic representation of ORF2 (A) and segment 6 (B) coding regions used in cloning.

FIG. 2 shows the electrophoresis gel of the fragments amplified from rotavirus segment 6 and astrovirus ORF2 nucleotide sequences.

FIG. 3 illustrates the cloning strategy used in the plasmid construction.

FIG. 4A shows the electrophoretic profiles of pOM187 (rotavirus) and pOM186 (astrovirus) plasmids before digestion with restriction enzymes.

FIG. 4B shows the electrophoretic profiles of pOM187 (rotavirus) plasmid after digestion with restriction enzymes PstI/NcoI and pOM186 (astrovirus) plasmid after digestion with restriction enzymes HindIII/NheI.

FIG. 5 shows the result of the rapid extraction of the probable recombinant plasmids.

FIG. 6A shows the restriction profile of pOM187 plasmid (rotavirus) digested with PstI/NcoI.

FIG. 6B shows the restriction profile of pOM186 plasmid (astrovirus) digested with HindIII/NheI.

FIG. 7A shows the induction curve of the protein expression with 0.5 mM of IPTG in bacterial cells of strain B121 (DE) containing control plasmid.

FIG. 7B shows the induction curve of the protein expression with 1 mM of IPTG in bacterial cells of strain B121 (DE) containing control plasmid.

FIG. 7C shows the induction curve of the protein expression with 2 mM of IPTG in bacterial cells of strain B121 (DE) containing control plasmid.

FIG. 8A shows the induction curve of the protein expression with 0.5 mM of IPTG in bacterial cells of strain B121 (DE) containing pOM187 plasmid (rotavirus).

FIG. 8B shows the induction curve of the protein expression with 1 mM of IPTG in bacterial cells of strain B121 (DE) containing pOM187 plasmid (rotavirus).

FIG. 8C shows the induction curve of the protein expression with 2 mM of IPTG in bacterial cells of strain B121 (DE) containing pOM187 plasmid (rotavirus).

FIG. 9A shows the induction curve of the protein expression with 0.5 mM of IPTG in bacterial cells of strain B121 (DE) containing pOM186 plasmid (astrovirus).

FIG. 9B shows the induction curve of the protein expression with 1 mM of IPTG in bacterial cells of strain B121 (DE) containing pOM186 plasmid (astrovirus).

FIG. 9C shows the induction curve of the protein expression with 2 mM of IPTG in bacterial cells of strain B121 (DE) containing pOM186 plasmid (astrovirus).

FIG. 10 shows the expressed protein localization experiment performed in strain BL21(DE) containing the pOM187 plasmid (rotavirus).

FIG. 11 shows the expressed protein localization experiment performed in strain BL21(DE) containing the pOM186 plasmid (astrovirus).

FIG. 12A shows the expression of rotavirus VP6 protein by induction of bacterial cells of strain B121 containing pOM187 plasmid.

FIG. 12B shows the expression of astrovirus VP90 protein by induction of bacterial cells of strain B121 containing pOM186 plasmid.

FIG. 13A shows the analysis result of the anti-histidine antibody-stained Western-blot of the recombinant rotavirus VP6 protein expressed by bacterial strain B121(DE) containing the pOM187 plasmid.

FIG. 13B shows the analysis result of the anti-GST antibody-stained Western-blot of the recombinant rotavirus VP6 protein expressed by bacterial strain B121(DE) containing the pOM187 plasmid.

FIG. 13C shows the analysis result of the anti-rotavirus (kit EIARA) antibody-stained Western-blot of the recombinant rotavirus VP6 protein expressed by bacterial strain B121(DE) containing the pOM187 plasmid.

FIG. 14A shows the analysis result of the anti-histidine antibody-stained Western-blot of the recombinant astrovirus VP90 protein expressed by bacterial strain B121(DE) containing the pOM186 plasmid.

FIG. 14B shows the analysis result of the anti-GST antibody-stained Western-blot of the recombinant astrovirus VP90 protein expressed by bacterial strain B121(DE) containing the pOM186 plasmid.

FIG. 15A shows the analysis result of the anti-histidine antibody-stained Western blot of the localization experiment performed in strain BL21(DE) containing the pOM187 plasmid (rotavirus).

FIG. 15B shows the Western blot analysis result of the localization experiment performed on the strain BL21(DE) containing the pOM187 plasmid (rotavirus) shown of the localization experiment performed with the strain BL21(DE) containing the pOM186 plasmid (astrovirus) revealed with anti-histidine antibodies.

FIG. 16B shows the analysis result of the anti-GST antibody-stained Western blot of the localization experiment performed in strain BL21(DE) containing the pOM186 plasmid (astrovirus).

FIG. 17 depicts the scheme of the IDEIA immunoenzymatic test.

FIG. 18 depicts the scheme of the EIARA immunoenzymatic test.

FIG. 19 illustrates the preparation of the inclusion bodies.

FIG. 20 shows the purified inclusion bodies.

FIG. 21 illustrates the affinity chromatography purification steps of the recombinant VP6 protein.

FIG. 22 illustrates the affinity chromatography purification steps of the recombinant VP90 protein.

FIG. 23 indicates the purified VP6 and VP90 recombinant proteins.

FIG. 24 indicates the SDS-PAGE electrophoresis quantification of the VP6 and VP90 recombinant proteins.

FIG. 25 indicates the assessment of the purified VP6 and VP90 recombinant protein homogeneity.

FIG. 26 indicates the Western-blot of the VP6 viral protein revealed using rabbit serum inoculated with the VP6 rotavirus recombinant protein.

FIG. 27 indicates the Western-blot of the VP6 protein revealed using rabbit serum inoculated with this same protein.

FIG. 28 indicates the Western-blot of the VP90 recombinant protein revealed using rabbit serum inoculated with this same protein.

FIG. 29 indicates the ELISA analysis of the recombinant anti-VP6 policlonal serum.

FIG. 30 indicates the assessment of the purified immunoglobuline homogeneity.

### DETAILED INVENTION DESCRIPTION

The present invention refers to the production of specific recombinant viral proteins, for application in the construction of a diagnostic kit for the simultaneous detection of the two main gastroenteric viruses. More specifically, the viruses detected by the kit are caused by rotavirus and astrovirus.

The present invention is described in detail through the examples presented below. It should be pointed out that the invention is not limited to these examples, but that it also includes variations and modifications within the limits in which it functions.

### Example 1

### Obtainment of pOM187 Vectors for Rotavirus and pOM186 for Astrovirus

RNA pattern molecules were extracted from fecal positive samples for the viruses caused by rotavirus and by astrovirus, through an appropriate commercial kit. The RNA pattern molecules were used for obtaining the cDNA.

In the present invention, for the obtainment of the cDNA, a selection of starting oligonucleotides sequences, which are organized in Table 1, was performed. This cDNA was obtained through transcriptase reverse reaction.

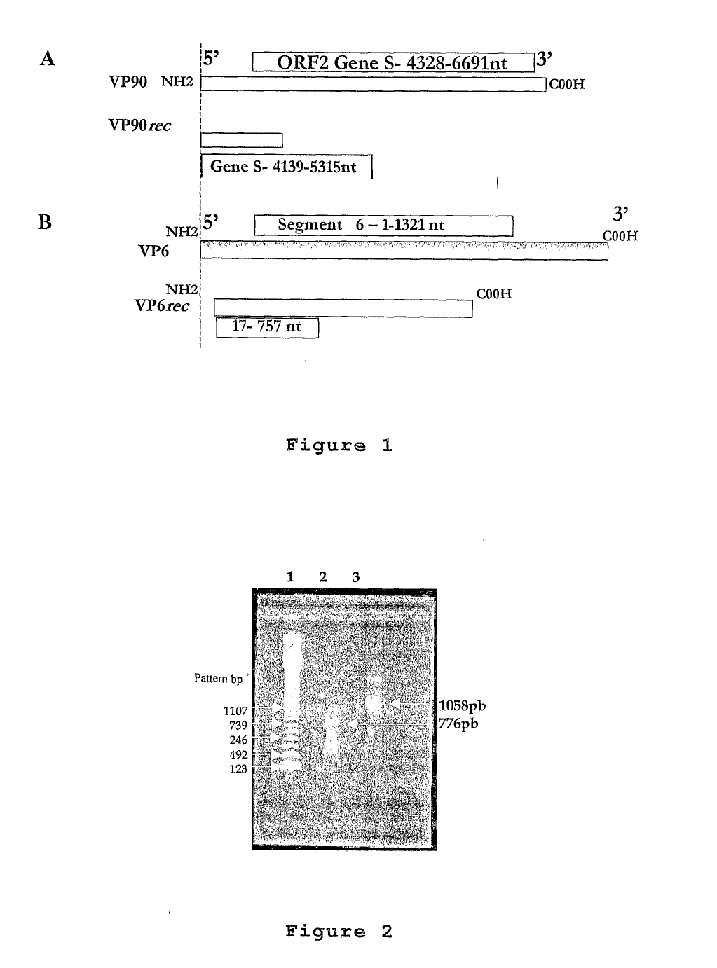

FIG. 1 shows the localization of the nucleotide sequences cloned in the virus genoma. The present concretization has as its base the segment 6 of the Wa human RNA rotavirus and ORF2 human type 1 RNA rotavirus.

TABLE 1
Starting Oligonucleotides used for the syn-
thesis of cDNA, in the amplification reaction
by RT-PCR

| | Genoma | |
|---|---|---|
| | localiza- | |
| Starters | tion | Sequence (5′-3′) |
| Positive | 17-42 | CTTCgCCATggAggTTCTgTACTCAC |
| rotavirus | | |
| Negative | 730-757 | gTCgCgCCATCggCCgAATTAATTACTC |
| rotavirus | | |
| Positive | 4139-4164 | AATCACTCCATgggAAgCTCCTATgC |
| astrovirus | | |
| Negative | 5315-5340 | gTgACAAgCTCggCCgCAgATACAgC |
| astrovirus | | |

Initially, the cDNA fragments obtained through the reverse transcriptase reaction were amplified by polymerase (PCR) chain reaction and recombined in an appropriate plasmodium vector as per example the pOM vector.

As a result of the recombination of these fragments in the pOM vector, two new vectors were obtained, which were denominated pOM144-rotavirus, which contains an insert of approximately 776 bp and pOM144-astrovirus, which contains an insert of approximately 1058 bp.

The result of the amplification through the PCR reaction to the DNA fragments is shown in FIG. 2, which shows—molecular weigh pattern of 123 bp (Sigma); 2—PCR reaction product for the rotavirus segment 6; 3—PCR reaction product for the ORF 2 of the astrovirus.

From these two new vectors, the DNA fragments were amplified again through PCR reaction. The starting oligonucleotides used in the second reaction were:

Sense:
5′-AggAAAAAAgCggCCgCCACCATGGCGAAACgCgCCAgACCgTC;
antisense:
5′- AggAAAAAACggCCgATgTTTgCAgggCTAgC.

The underlined bases represent the restriction sites of the NcoI and XmaIII endonucleases, respectively.

The PCR reaction products were purified through the use of a commercial kit. The present concretization used a NucleoSpin 2 in 1 kit.

The mentioned PCR reaction products were submitted to enzymatic reactions using the restriction endonucleases NcoI and XmaIII. Next, they were subcloned in a new pOM vector. These new vectors were denominated pOM32-rotavirus and pOM32-astrovirus.

The pOM32-rotavirus and pOM32-astrovirus, which contain the restriction sites of the NcoI and MluI endonucleases, were submitted again to enzymatic reactions using restriction endonucleases NcoI and MluI.

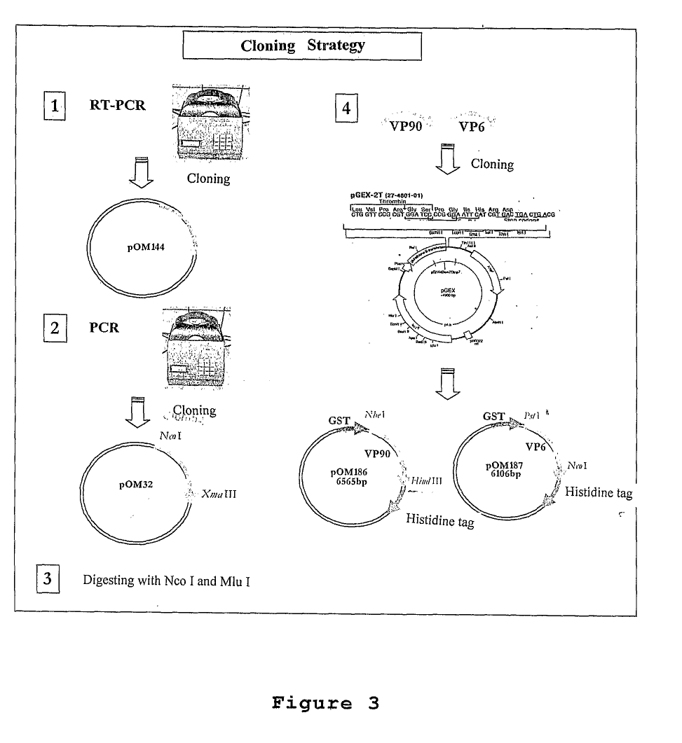

The fragments released through the enzymatic reaction were purified and again subcloned in an appropriate expression vector, as for example the pGEX-NX-2T. The new expression vectors obtained were denominated pOM187 for the rotavirus and pOM186 for astrovirus. FIG. 3 shows the cloning procedure used in the present concretization.

### Example 2

### Bacterial Transformation and DNA Plasmodial Acquisition with High-Level Purity

The E. coli bacterial strain was transformed, with the pOM186 and pOM187 expression vectors, through the methodology of electroporation. This electroporation was performed so as to submit the bacterial cells to an electrical discharge of 2500 volts for 5 seconds. In the present concretization the TOP10F′ E. coli strain is used. After the discharge, recovery of the bacterial cells was done through the addition to the reaction of approximately 500 μl of an adequate growth medium. In the present concretization, a liquid SOC was used [2% of triptone, 0.5% of yeast extract, 8.6 mM of NaCl, 2.5 mM of KCl, 20 mM of MgSO₄ and 20 mM of glucose, pH 7.0]. After the addition of the liquid SOC, the reaction was incubated at 37° C. temperature under agitation (approximately 250 rpm) for approximately an hour.

After the agitation period, the reaction plaquing was performed in two Petri dishes with 20 ml of an adequate medium, as for example the LB-solid medium [Luria Bertani:1% de triptone, 0.5% of yeast extract, 1% of NaCl, pH 7.0 (liquid-LB) with 1.5% (p/v) of agar], which was supplemented with ampicillin to a preferential concentration of 100 μg/mL. In the first Petri dish approximately 20% of the reaction was plaqued in a solid LB medium. In a second Petri dish, approximately 80% of the reaction was plaqued in a solid LB medium.

The two Petri dishes were kept for approximately 16 hours at a chosen temperature of 37° C. After this period the incubation period of the two Petri dishes was observed to evaluate the bacterial growth colonies. Approximately 10 bacterial growth colonies, resistant to ampicillin, were removed from the plates containing 20% to 80% of the bacterial transformation (5 colonies in each dish) and were transferred to appropriate tubes, which contain the liquid LB medium supplemented with an antibiotic, as for example the ampicillin, in a chosen amount of 100 μg/mL. After the transfer, the tubes were incubated for approximately 16 hours under constant agitation (approximately 150 rpm) at a preferential temperature of 37° C.

The plasmodium DNA of the cultivated E. coli strain was extracted using a commercial kit, as for example “Concert Rapid Plasmid Purification Systems” (Invitrogen-USA), in accordance with the manufacturer's recommendations, in order to obtain this plasmodium DNA with a high purity level.

The purified plasmodium DNA was quantified through an estimate of approximate quantity, in accordance with the specific DNA band submitted to the electrophoresis technique in 0.8% in agar gel in an adequate buffer solution, as for example a TBE 1× solution (89 mM Tris-borate, 2 mM EDTA pH 8.0). The agar gel was dyed with an adequate solution. In the present concretization, for the gel to be dyed, a solution of etidium bromide in a preferential concentration of 0.5 μg/mL was added, for later visualization under UV light (U.V.).

### Example 3

### Characterization of the Plasmodium pOM186 and pOM187 Concretizations

The final plasmodium constructions were characterized through the enzymatic breaking reaction with PstI/NcoI endonuclease restriction reactions for the VP6 genes of the rotavirus (pOM187) and HindIII/NheI for the VP90 of the astrovirus (pOM186).

The digestion reactions of the plasmodium DNAs comprehends the following components and steps:
approximately 20 μl of a solution containing 1 μg/μl of the plasmodium constructions pOM187 and pOM186 purified from E. coli TOP10F′ strain,
approximately 1 μl of each one of the restriction enzymes PstI/NcoI (100/μl each) for the VP6 genes of the rotavirus,
approximately 1 μl of each one of the restriction enzymes HindIII/NheI (10 U/μl each) for the VP90 genes of the astrovirus,
addition of approximately 2.5 μl of a commercial buffer solution recommended for each one of the HindIII/NheI and PstI/NcoI enzymes used, where the mentioned buffer solution must be preferentially 10 times the concentration,
addition of water (q.s.p), to obtain a final volume of 25 μl,
incubation of the reaction for approximately 2 hours at a preferential temperature of 37° C., in a specific equipment, as for example, a hot bath.

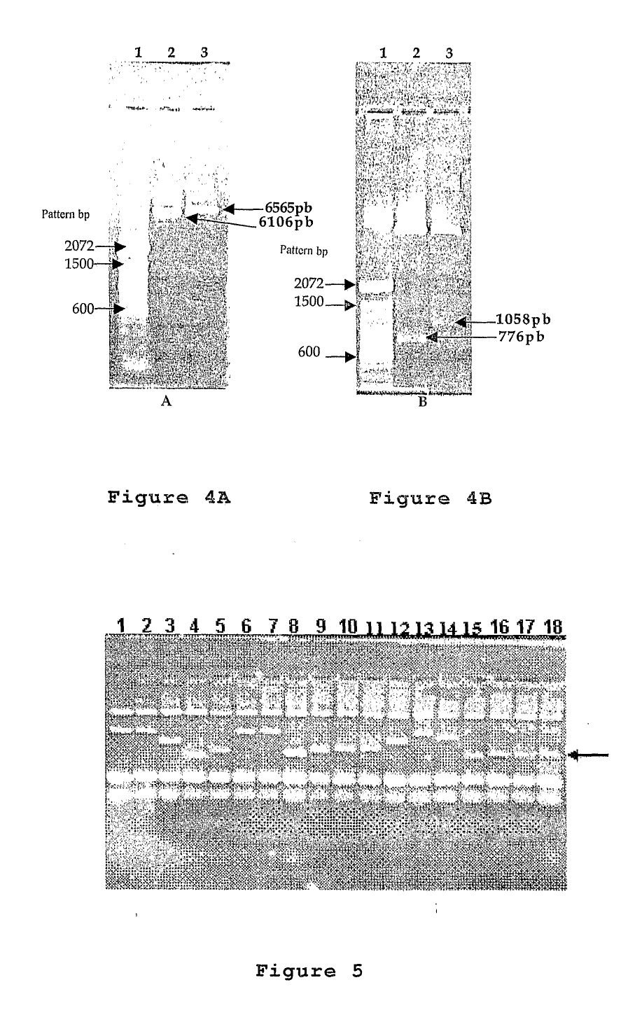

After the digestion reaction, in which enzymatic rupture occurred, the reaction product was submitted to electrophoresis gel as described in example 2. FIG. 4A and FIG. 4B show the electrophoresis profile of DNA after the digestion reaction of the plasmodium constructions pOM187 and pOM186, respectively. For figure A: 1—Molecular weight pattern of 100 bp (Invitrogen); 2—non-digested pOM187; 3—non-digested pOM186. For figure B: 1—Molecular weight pattern of 100 bp (Invitrogen); 2—digested pOM187; 3—digested pOM186.

The double digestion reaction with the restriction endonucleases present an electrophoresis profile, in which both digested constructions have bands: one correspondent to the vector and the another correspondent to the insert.

In order to construct the pOM 187, the bands present a size of approximately 5330 bp for the vector and a size of 776 bp for the insert, while the construction of the pOM186 presents a band for the vector with an approximate size of 5507 bp and another band with an approximate size of 1058 bp for the insert.

### Example 4

### Bacterial Transformation

After the characterization of the pOM186 and pOM187 plasmodium constructions, they were used in a new bacterial transformation. Reliable bacterial cells of the E. coli strain, which were previously prepared by a CaCl₂ method, were used for this experiment. In the present concretization cells of E. coli from the B121DE were used.

The bacterial cells of the B121DE strain used were initially unfrozen in an ice bath. After this procedure, approximately 1 μg of purified plasmodium was added to the bacterial cells and the reaction was incubated for its processing in an ice bath for approximately 30 minutes.

After 30 minutes, the reaction was submitted to a thermal impact for approximately 70 seconds at a temperature of 42° C. Afterwards, a new incubation for the conclusion of the transformation process was performed in an ice bath for approximately 10 minutes.

The recovery of bacterial cells was performed through the addition of approximately 300 μl of an appropriate medium to the reaction. In the present concretization the liquid Lb medium was used. After the addition, the reaction was incubated at a temperature of 37° C. under agitation (approximately 250 rpm) for approximately one hour.

After the agitation time, plaquing of the reaction was performed. It occurred as described in Example 2.

In one of the Petri dish, approximately 10% of the reaction was plaqued in solid LB medium. In another Petri dish, approximately 90% of the reaction was plaqued in solid LB media. The bacterial growth was analyzed as described in Example 2.

After the period for bacterial growth, an extraction of plasmodium DNA was performed, so the presence of bacterial clones of the plasmodium resistant to ampicillin could be evaluated.

### Example 5

### Selection and Purification of plasmodium DNA

In this evaluation procedure, in each one of the tubes where bacterial growth had occurred, plasmodium DNA was extracted, through the fast pace phenol/chloroform technique. The material obtained from the extraction was analyzed through the electrophoresis technique in agar gel as described in example 2. FIG. 5 shows the result of this technique for fast pacing extraction of plasmodium. To the FIG. 5, lines 1 to 17 show: Probable recombinant plasmodium, line 18: Plasmodium control without insert. As it can be observed, plasmodium from lines 1, 2, 3, 6, 7, 12, 13 and 14 show a size greater than the control (arrow). The plasmodium selection occurred through the comparison between the plasmodium with insert with the one without insert. Plasmodia larger than the plasmodium without insert (control) were selected for further analysis.

In FIG. 5, it can be observed that for both plasmodium constructions, all selected colonies tend to present a recombinant plasmodium.

After a bacterial colony selection, each one of the liquid cultures developed from bacterial recombinant clones selected through a plasmodium fast pace extraction were extracted through an appropriate kit, as for example the “Concert Rapid Plasmid Purification Systems” (Invitrogen-USA), in accordance with the manufacturer's recommendation and analyzed through the digestion reaction with the restriction endonucleases which is similar to the reaction performed for the characterization of plasmodium constructions pOM187 and pOM186 purified from the TOP10F′ strain of the E. coli.

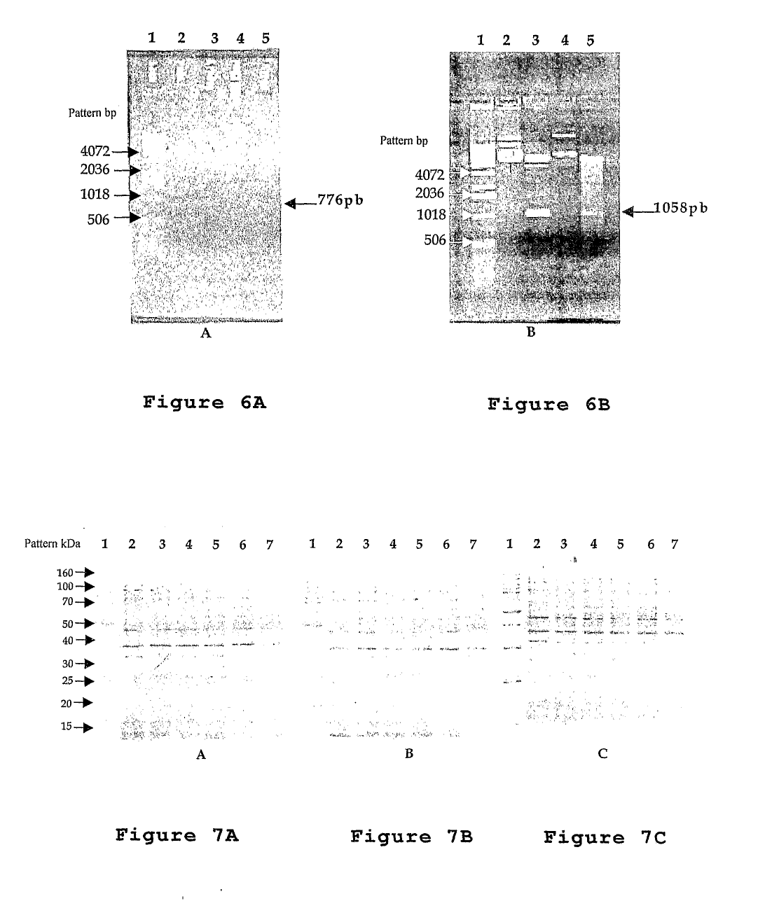

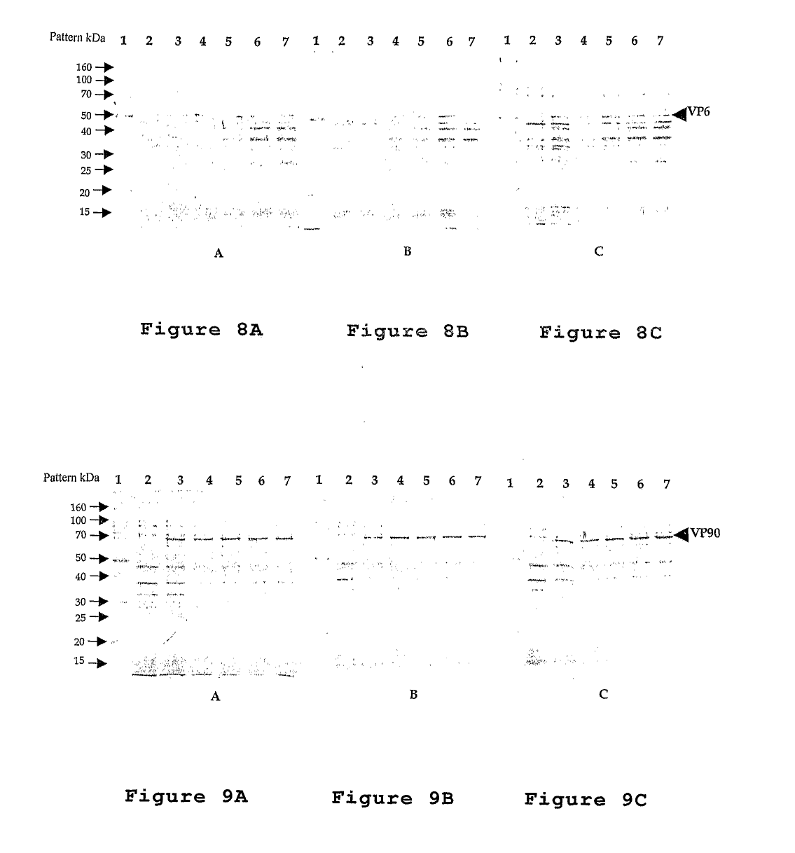

FIG. 6A and FIG. 6B show a non-digested purified plasmodium and the restriction profile with the PstI/NcoI enzymes for the pOM187 plasmodium (rotavirus) and with the HindIII/NheI enzymes for the pOM186 plasmodium (astrovirus) respectively, in comparison with the results obtained in the digestion of the control plasmodium extracted from the TOP10F′ strain. The above mentioned Figures show in A: 1—Molecular weight pattern of 1 kB (Invitrogen); 2—non-digested pOM187 purified from strain TOP10F′; 3—digested pOM187 purified from strain TOP10F′; 4—non digested pOM187 purified from strain B121(DE); 5—digested pOM187 purified from strain B121(DE). For B: 1—Molecular weight pattern of 1 kB (Invitrogen); 2—non-digested pOM186 purified from strain TOP10F′; 3—digested pOM186 purified from TOP10F′; 4—non-digested pOM186 purified from strain B121(DE); 5—digested pOM186 purified from strain B121(DE).

In the electrophoresis profile for the pOM 187 construction, the bands present an approximate size of 5330 bp for the vector and an approximate size of 776 bp for the insert, while the pOM186 construction presents a band of approximately 5507 bp and a band with an average size of 1058 bp for the insert. The performed comparison demonstrates that the same electrophoresis band profile was observed for both the insert and vector.

### Example 6

### Automatic Nucleotide Sequencing of Plasmodium Vectors

Once the recombinant clones were purified, the automatic nucleotide sequencing of plasmodium vectors was performed.

For the nucleotide sequencing of the plasmodium vectors, a commercial kit was used for the preparation of the reactions, in which dideoxynucleotide is traced with a fluorescent molecule, as per the manufacturer's instructions. In the present concretization the commercial kit used was the “ABI Prism Big Dye Terminator Cycle Sequencing Ready Reaction Kit” 3.1 version. The automatic sequencing and reading were performed in appropriate equipments, which can be used for both the analysis and generation of electrophoretograms, as for example the ABI PRISM Genetic Analyser.

For the visualization and impression of electrophoretograms obtained in the automatic sequencing an adequate program was used, as for example the Chromas.

For the analysis, the sequences obtained in the electrophoretograms were compared with standard sequences of the segment 6 regions of the Wa human rotavirus RNA and the ORF2 of the human type 1 astrovirus RNA. Such standard sequences are available in the Gene Bank of the National Center for Biotechnology Information (NCBI).

Such comparison was performed through the alignment of the sequences using an adequate program sequence, as for example the NCBI Blast program. This comparison demonstrates that the cloned sequences do not present nucleotide alterations and confirm that the cloning material truly corresponds to the target regions of the invention, which corresponds to the nucleotide positions 23 to 742(VP6) and nucleotide positions 4145 to 5325(VP90).

### Example 7

### Construction of the Induction Curve

For the standardization of the important parameters necessary for the expression of recombinant proteins the construction of an induction curve was performed. For this construction, the bacterial cultures of BL21(DE) strain, which contain the expression vectors pOM187, pOM186 and the plasmodium control (without insert), were transferred to adequate tubes, through the transplantation technique. Such tubes contained approximately 5 mL of medium, as for example, the liquid LB medium supplemented with ampicillin at approximately 50 μg/mL. Such tubes were incubated at a temperature of 37° C. for approximately 16 hours under constant agitation (150 rpm).

After the incubation period, at least three transfers were performed, through the transplantation technique, of each one of the bacterial cultures that contained expression vectors. Approximately 100 μl of each bacterial culture media was transferred to a new 50 mL test tube that contained a final volume of approximately 10 mL of liquid LB medium supplemented with ampicillin at approximately 50 μg/mL.

The new cultures were grown under vigorous agitation (approximately 250 rpm) at a temperature of 37° C. until the reading of these cultures in a spectrophotometer reached a minimum 0.4 D.O. In the present concretization the reading in spectrophotometer occurred at a wavelength of 550 nm.

An approximate sample of 1 mL from each of the bacterial cultures was collected and identified as a zero starting time (TO) or non-induced control culture. To the remaining bacterial cultures an inducing substance was added, as for example the isopropil-b-D-tiogalactopiranosideo (IPTG) inducer.

The IPTG concentrations were different for each of the remaining bacterial cultures. In the present concretization the IPTG concentrations used were 0.5 mM, 1 mM e 2 mM. After the addition of IPTG, the bacterial cultures were again incubated under vigorous agitations (approximately 250 rpm) for a maximum period of 5 hours.

During the incubation period, each hour after the starting time, a new sample of approximately 1 mL was collected and identified, as described in table 2.

TABLE 2
Sampling scheme of the recombinant bacterial
cultures induced during a 5 hours period

| | Sample | IPTG concentration | | | | | |
|---|---|---|---|---|---|---|---|
| | description | 0.5 mM | | 1 mM | | 2 mM | |
| Time | T0 | Zero | hour | Zero | hours | Zero | hours |
| | T1 | 1 | hour | 1 | hour | 1 | hour |
| | T2 | 2 | hours | 2 | hours | 2 | hours |
| | T3 | 3 | hours | 3 | hours | 3 | hours |
| | T4 | 4 | hours | 4 | hours | 4 | hours |
| | T5 | 5 | hours | 5 | hours | 5 | hours |

All collected samples were D.O. evaluated in a spectrophotometer at a wavelength of 550 nm. For D.O. readings above 1.0, the cultures were diluted 2 to 10 times. The dilution degree was dependent of the obtained bacterial growth. After the dilution, a new evaluation of the D.O in spectrophotometer at a 550 nm wavelength was performed.

All collected samples from the bacterial cultures were centrifuged preferentially at 8.500×g for approximately 5 minutes. The supernatants of the bacterial cultures after centrifugation were discarded. The obtained precipitate was frozen for further analysis under the electrophoresis technique in polyacrylamide gel.

The procedure for analysis of products occurred so that all frozen precipitates were re-suspended in solution in a buffer solution of 2× sample [50 mM Tris-HCl pH6.8, 100 mM of DTT, 2% of SDS, 0.1% of blue of bromophenol, 10% of glycerol].

The solution volume of the buffer sample, in which the precipitates were re-suspended, varied according to the D.O. of each sample obtained during the experiment of the induction curve. In the present concretization, the amount of buffer sample added followed the rate that for each 0.1 of D.O. 10 μl of the buffer solution were added to the sample 2×.

After the precipitates were re-suspended, the samples were warmed up at a temperature band of 95 to 105° C. for 5 minutes. After this, the samples were centrifuged at 8.500×g for approximately 30 seconds.

### Example 8

### Analysis Through the SDS-Page Technique

The prepared samples, as mentioned in example 7, were analyzed through the electrophoresis technique in polyacrylamide denaturant gel SDS-PAGE type. Such gel was prepared at a concentration of 10% for the separating gel phase and then submitted to an electrophoresis run at 100 volts, for approximately 2 hours in an adequate buffer, as for example the buffer of the 1× run [50 mM of Tris pH 8.3, 384 mM of glycine, 0.1% of SDS].

FIGS. 7A, 7B, 7C, 8A, 8B, 8C, 9A, 9B, 9C and tables 3, 4 and 5 show results from time and inducer concentration curves realized with the B121(DE) strain transformed with the plasmodium concentrations pOM187, pOM186 and plasmodium control (without insert). In accordance with the observed figures, it was observed that there was no significant difference between the induction times and the inducer concentrations.

In this way, there occurred a standardization, in which the induction experiments were performed using 1 mM of IPTG and that the induction time would be of approximately 4 hours. For the A figures—Induction curve prepared with 0.5 mM of IPTG; For the B figures—Induction curve prepared with 1 mM of IPTG; C figures—Induction curve with 2 mM of IPTG: 1—standard Molecular weight BenchMark (Invitrogen); 2—Bacterial culture non induced; 3—Bacterial culture induced for 2 hours; 4—Bacterial culture induced for 2 hours; 5—Bacterial culture induced for 3 hours; 6—Bacterial culture induced for 4 hours; 7—Bacterial culture induced for 5 hours.

TABLE 3
Evaluation of D.O. in spectrophotometer at a
550 nm wavelength of the samples of the recombinant
bacterial culture transformed with plasmodium control
collected during the construction of the induction curve.

| | Sample | IPTG Concentration | | |
|---|---|---|---|---|
| | Description | 0.5 mM | 1 mM | 2 mM |
| Time | T0 | 0.781 | 0.802 | 0.815 |
| | T1 | 1.832 | 1.906 | 1.958 |
| | T2 | 2.84 | 3.235 | 3.295 |
| | T3 | 4.96 | 5.115 | 5.195 |
| | T4 | 5.67 | 5.82 | 5.75 |
| | T5 | 6.77 | 6.59 | 6.44 |

TABLE 4
Evaluation of D.O. in spectrophotometer at a
550 nm wavelength of the samples of the recombinant
bacterial culture transformed with plasmodium pOM187
collected during the construction of the induction curve.

| | Sample | IPTG concentration | | |
|---|---|---|---|---|
| | Description | 0.5 mM | 1 mM | 2 mM |
| Time | T0 | 0.444 | 0.458 | 0.483 |
| | T1 | 0.844 | 0.828 | 0.848 |
| | T2 | 1.145 | 1.1 | 1.12 |
| | T3 | 1.225 | 1.23 | 1.27 |
| | T4 | 1.25 | 1.26 | 1.275 |
| | T5 | 1.285 | 1.38 | 1.35 |

TABLE 5
Evaluation of D.O. in spectrophotometer at a
550 nm wavelength of the samples of the recombinant
bacterial culture transformed with plasmodium Pom186
collected during the construction of the induction curve.

| | Sample | IPTG concentration | | |
|---|---|---|---|---|
| | Description | 0.5 mM | 1 mM | 2 mM |
| Time | T0 | 0.735 | 0.749 | 0.761 |
| | T1 | 1.444 | 1.464 | 1.448 |
| | T2 | 1.83 | 1.815 | 1.855 |
| | T3 | 2.06 | 2.0 | 2.08 |
| | T4 | 2.02 | 2.035 | 2.085 |
| | T5 | 2.425 | 2.43 | 2.525 |

### Example 9

### Localization Experiment of the Recombinant Protein

After the construction of the induction curve, a localization curve experiment was performed, so the expression site of each recombinant protein could be identified. For this experiment, each bacterial culture from the BL21(DE) strain, which contain the expression plasmodium, was submitted to a induction reaction.

The procedure for such reaction involved: a medium, in which 1 mL of grown pre-inoculate was added to approximately 100 mL of liquid LB medium supplemented with 50 μg/mL of ampicillin. Such culture was kept under vigorous agitation under the same conditions described in the example 7 until the spectrophotometer reading reached a minimum D.O. of 0.4. The volume of the utilized culture for this experiment was 50 mL.

After the induction procedure, the separation of the exported proteins was performed; these are proteins that are released to the medium where the bacteria were grown. For this procedure, the bacterial cell culture was centrifuged for approximately 15 minutes at approximately 6.500×g.

After the centrifugation, approximately 1 mL of supernatant was transferred to a 1.5 mL test tube to which 100 μl of trichloroacetic acid (TCA) 100% was added. This solution was vigorously agitated, kept in an ice bath for approximately 15 minutes and centrifuged at approximately 14.000×g during 15 minutes. After this centrifugation, the precipitate was washed with 100 μl of acetone and rapidly centrifuged at approximately 14.000×g. The supernatant was discarded and the dried precipitate was re-suspended with 100 μl of a PBS solution (137 mM of NaCl, 2.7 mM KCl, 10 mM Na₂HPO₄ and 1.8 mM KH₂PO₄) 1×pH 7.4 and approximately 100 μl of a buffer solution of sample 2×.

Afterwards, the protein solution was heated at a temperature band of 95-105° C. for approximately 5 minutes. Then, the solution was frozen for further analysis through electrophoresis technique in SDS-PAGE gel, as described in Example 8.

For the separation of the periplasmatic proteins, the bacterial precipitate obtained after centrifugation was re-suspended in 30 mL of an adequate buffer solution. The present concretization used a Tris-HCl 30 mM pH 8.0 buffer solution, which contained approximately 20% of sacarose and 1 mM of EDTA.

Such cellular suspension was kept under constant mechanical agitation for approximately 10 minutes at a temperature band of 23-26° C.

After the agitation period, the cellular suspension was centrifuged at 10.000×g for approximately 10 minutes. The supernatant was discarded and the bacterial precipitate was re-suspended in an adequate solution, as for example, a cold MgSO₄ 5 mM solution. The cellular suspension was mechanically agitated for 10 minutes in ice, in order to allow the transfer of the periplasmatic proteins to a buffer solution. After the agitation period the cellular suspension was again centrifuged at 10.000×g for 10 minutes. After the centrifugation, approximately 1 mL of the supernatant was transferred to a test tube of 1.5 mL and then, 100 μl of trichloroacetic acid (TCA) 100% was added. This solution was agitated vigorously, incubated in ice bath for 15 minutes and centrifuged at 14.000×g for 15 minutes.

After this centrifugation, the obtained precipitate was washed with approximately 100 μl of acetone and rapidly centrifuged at 14.000×g. The supernatant was discarded and the dried precipitate was re-suspended in 100 μl of a PBS 1× solution and approximately 100 μl of a buffer solution of sample 2×. Subsequently, the protein solution was heated at a temperature band of 95-105° C. for approximately 5 minutes. The heated solution was frozen for further analysis through the electrophoresis technique in SDS-PAGE gel, as described in Example 8.

For the preparation of the cytoplasmatic proteins, the bacterial precipitate obtained after the last centrifugation of the step described above was re-suspended in approximately 4 mL of an adequate buffer solution of cold Tris-HCl 20 mM pH 7.5 solution for further lysis of the bacterial cells.

The release of the cytoplasmatic protein material was performed after the bacterial lysis with lysozyme. The described procedure occurred with the addition of lysozyme at a 100 μg/mL concentration of the bacterial culture, at a preferential temperature of 30° C. for approximately 15 minutes. After the addition of the lysozymes, for the procedure of cellular lysis to be complete, a cellular fragmentation with ultra sound was performed.

The cellular suspension that was in ice was submitted to an ultrasound process performed in 3 cycles of 2 minutes with constant pulse. The gap between each one of the cycles was of approximately 10 minutes.

For the separation of the soluble from the insoluble fraction approximately 1.5 mL of lysed bacterial cells was centrifuged at 14.000×g for approximately 10 minutes. The supernatant of the sample was transferred to a 1.5 mL test tube. Approximately 100 μl of this supernatant was transferred to a 1.5 mL test tube that contained 100 μl of a buffer solution of sample 2×. After the addition of the supernatant, the buffer solution was heated at a temperature band of 95-105° C. for approximately 5 minutes and then frozen for analysis through the electrophoresis technique in SDS-PAGE gel as described in example 8.

The precipitate formed in the centrifugation of lysed bacterial cells corresponds to the insoluble fraction of the sample, denominated included bodies. This precipitate was rinsed at least twice with an adequate buffer solution, as for example a Tris-HCl 20 mM pH 7.5 solution, where, at the end of each rinsing, the sample was again centrifuged. Next, the obtained precipitate after the last centrifugation rinsing process was re-suspended in approximately 1.5 mL of an adequate solution, as for example, a SDS 1% solution.

After the re-suspension of the precipitate in a SDS solution, approximately 100 μl of this suspension was transferred to a tube, and 100 μl of a buffer solution of sample 2× was added. Subsequently, the suspension was heated at a temperature band of 95-105° C. for approximately 5 minutes. The protein solution was then frozen for further analysis through the electrophoresis technique in SDS-PAGE gel as described in example 8.

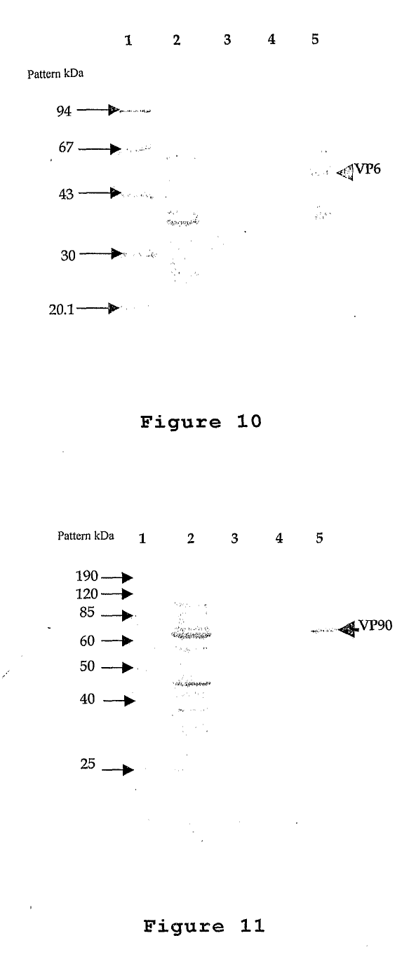

FIGS. 10 and 11 show that the recombinant proteins are in insoluble inclusion bodies in the cytoplasm of bacterial cells. For FIGS. 10 and 11, there is for 1—pre-dyed standard of low molecular weight (GE Healthcare); 2—induced bacterial culture for 4 hours; 3—periplasmatic fraction; 4—Cytoplasmatic culture; 5—Inclusion bodies.

### Example 10

### Expression of Recombinant Proteins

For obtaining recombinant proteins in low scale, the following procedure was performed:

Such procedure consisted of an initial preparation of a bacterial transplantation denominated pre-inoculate, which consisted 5 mL of an adequate medium, as for example the liquid LB media, with 50 μg/mL of ampicillin which was inoculated in separate tubes with 20 μL of each one of the bacterial cultures, which contained recombinant plasmodia, pOM187 or pOM186. Such tubes were incubated at a temperature of 37° C. for 16 hours under constant agitation (approximately 150 rpm).

After the incubation stage, approximately 1 mL of grown pre-inoculate was added to approximately 100 mL of supplemented liquid LB medium in order to produce a new bacterial culture to contain recombinant plasmodia.

This culture was kept under vigorous agitation (approximately 250 rpm) at a temperature of 37° C. and submitted to a reading in the spectrophotometer as described in example 7.

When the desired D.O was reached, a sample of approximately 1 mL was collected from the culture. Such sample was centrifuged for approximately 15 minutes at approximately 8.500×g and was identified as a non-induced culture or control culture. The supernatants were discarded. The precipitates were frozen for further analysis through electrophoresis technique in SDS-PAGE gel as described in example 8.

To the remaining grown culture, IPTG 1 mM was added. Said culture was kept in vigorous agitation for approximately 4 hours (approximately 250 rpm).

At the end of the induction period, a new sample of approximately 1 mL was collected. This sample was evaluated as described in example 7. For reading above 1.0, the culture was diluted from 2 to 10 times. The dilution level of the sample was dependent on the obtained bacterial growth. A new D.O. evaluation at 550 nm wavelength in the spectrophotometer was performed.

After the reading, said sample was centrifuged for approximately 15 minutes at 8.500×g. The supernatant of the bacterial cultures was discarded. The obtained precipitate was frozen for further analysis through electrophoresis technique in SDS-PAGE gel, as described in example 8. This sample was identified as an induced culture.

The remaining volume of the culture was centrifuged for approximately 15 minutes at approximately 6.500×g. The supernatant was discarded and the precipitate was frozen for further processing.

The previously frozen precipitates were once again suspended in a buffer solution of sample 2×. The volume of the sample buffer solution where the precipitates were re-suspended varied according with the D.O. of each sample obtained during the induction experiment. For each 0.1 D.O, approximately 10 μl of sample 2× buffer solution was added.

Once the precipitates had been re-suspended, the samples were heated at a temperature of approximately 95 to 105° C. for approximately 5 minutes. After heating, such samples were centrifuged at 8.500×g for approximately 30 seconds.

The samples prepared as described above were analyzed using the electrophoresis technique in SDS-PAGE gel as shown in example 8.

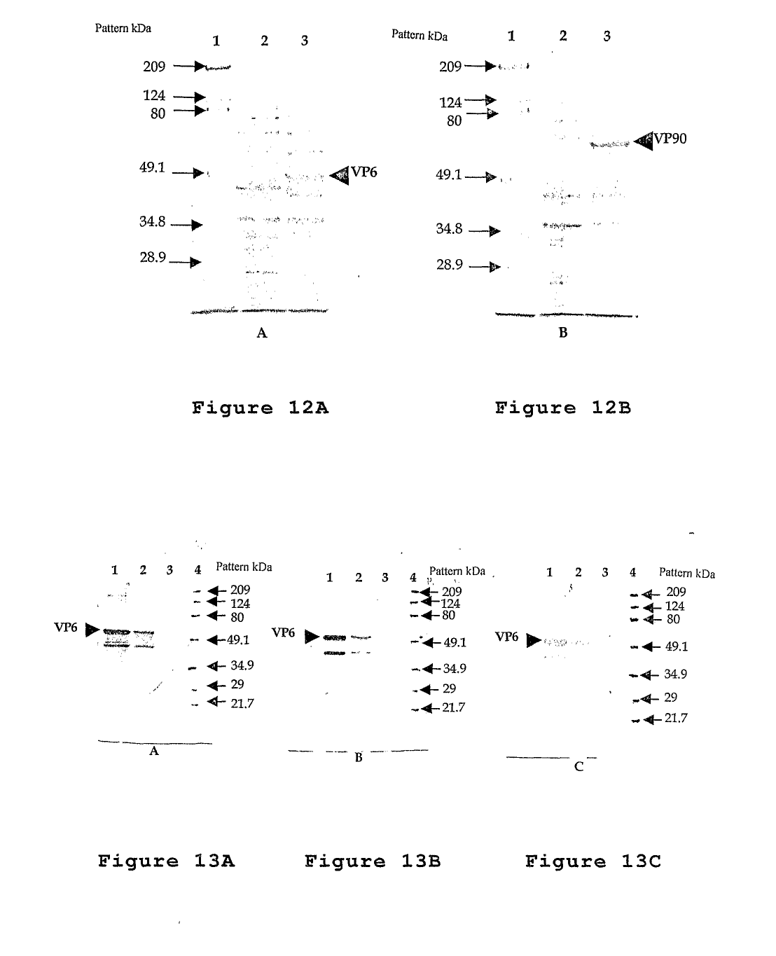

FIG. 12 A and FIG. 12B show the induction results obtained for the recombinant proteins VP6 and VP90, respectively. This figure shows: 1—pre-stained standard molecular weight (Bio-Rad); 2—Non induced bacterial culture; 3—Bacterial culture induced for 4 hours.

### Example 11

### Analysis of Antigenicity of the Expressed Recombinant Proteins

The analysis using the Western-blot technique was performed for the analysis of antigenicity of the expressed recombinant proteins. After being submitted to the electrophoresis technique, the proteins were transferred to nitrocellulose membranes using a Transbolt system. After the protein transfer to the nitrocellulose membranes, said membranes were blocked with an adequate blocking solution for approximately 16 hours, at a temperature of approximately 4° C. The blocking solution used in this operation was a PBS 1× solution containing approximately 0.05% (v/v) Tween 20, 5% (p/v) Molico milk.

Next, each one of the nitrocellulose membranes was incubated separately with one of the following antibodies: anti-histidine produced in mice, anti-GST antibody, which was produced in a goat and specific polyclonal antibody (anti-rotavirus produced in goats for the EIARA kit, BioManguinhos). Said antibodies were diluted in a blocking buffer solution according to the manufacturer's recommendation.

The nitrocellulose membranes were incubated with these solutions containing the antibodies for approximately two hours with light agitation at a temperature of approximately 25° C.

The nitrocellulose membranes were washed at least three times with a PBS 1× solution containing approximately 0.05% Tween 20. Each washing operation was performed so as to allow the membranes to be incubated for approximately ten minutes with said washing solution.

After at least three consecutive washing operations, an anti-mouse antibody or an anti-goat antibody together with alkaline phosphatase diluted in a blocking buffer solution was added, according to the manufacturer's recommendation.

The membranes were incubated with the mentioned solutions containing the antibodies, for, at least, 1 hour at a temperature of approximately 23-25° C. under soft agitation.

After the incubation period, the membranes were once again washed three times, at least, with a PBS 1× solution, containing approximately 0.05% Tween 20. Each washing operation was performed so as to incubate the nitrocellulose membranes for approximately 10 minutes in said washing solution.

After being washed with the PBS 1× solution, the nitrocellulose membranes were washed, at least, once with a 100 mM Tris-HCl ph 9 buffer solution containing 150 mM NaCl and 1 mM CaCl2. The revelation of the nitrocellulose membranes was done through the addition of NBT/BCIP (Promega) substrate to said buffer solution as mentioned above according to the manufacturer's recommendations.

All of the Western-blot performed to assess the antigenicity of the recombinant proteins expressed by B121 (DE) bacterial strain, supplied positive results as recognized by the anti-GST and anti-histidine antibodies and by specific anti-rotavirus antibody.

FIGS. 13A, 13B e 13C show the results of the assessment of the antigenicity of the recombinant VP6 rotavirus protein expressed by B121(DE) bacterial strain developed with anti-histidine antibody, anti-GST antibody and anti-rotavirus antibody, respectively.

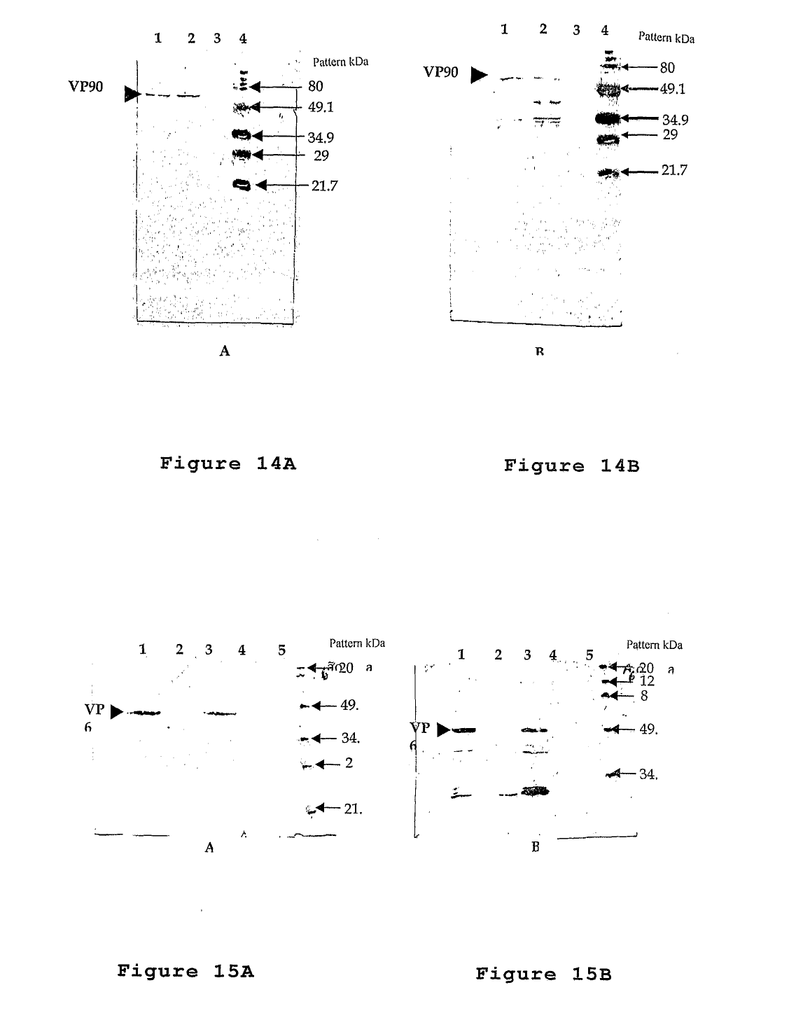

FIGS. 14A and 14B show the recognition of the astrovirus VP90 recombinant protein expressed by the B121(DE) bacterial strain developed with the anti-histidine antibody and with anti-GST antibody, respectively.

For FIGS. 13A, 13B,13C, 14A and 14B, we have: 1. isolated inclusion bodies; 2. Induced bacterial extract; 3. Non induced bacterial extract; 4. pre-stained standard molecular weight molecular (Bio-Rad).

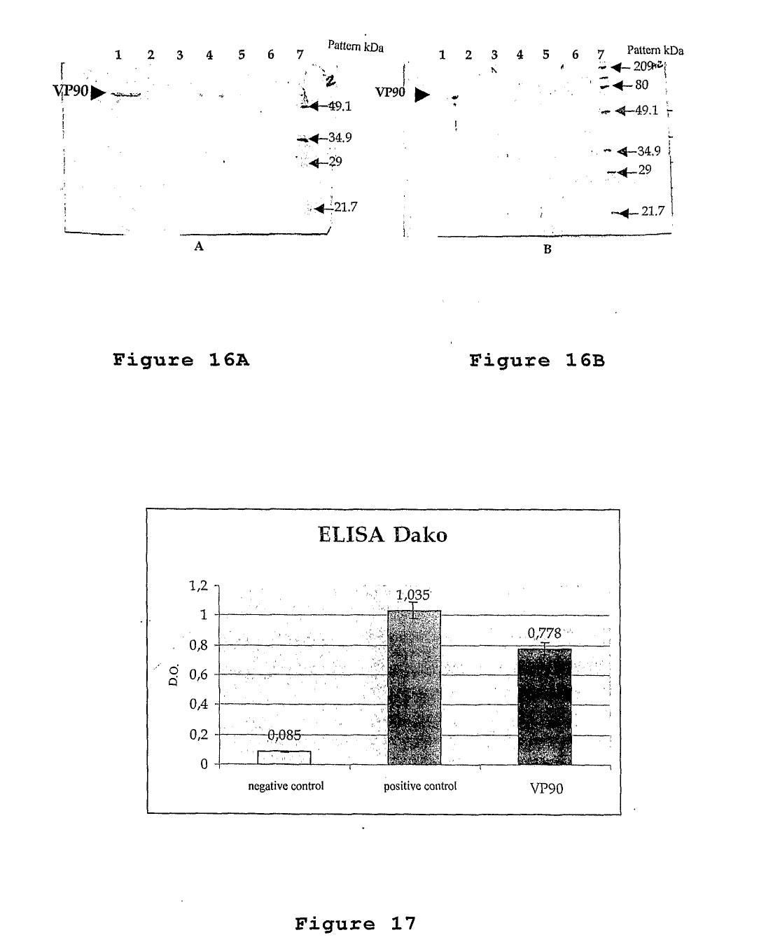

FIGS. 15A and 15B show the localization experiment performed with the BL21(DE) strain, which contains the pOM187 (rotavirus) plasmid developed with the anti-histidine antibody and with the anti-GST antibody, respectively. FIGS. 16A and 16B show the localization experiment with the strain BL21(DE), which contain the plasmid pOm186 (astrovirus) developed with antibody anti-histidine and with antibody anti-GST respectively.

For FIGS. 15A, 15B, 16A e 16B, we have: 1—Inclusion bodies; 2—citoplasmatic fraction; 3—Periplasmatic fraction; 4—induced bacterial culture 5—non induced bacterial culture; 6—Induced Bacterial Culture without plasmid; 7—pre-stained standard molecular weight (Bio-Rad).

Another method for assessing the antigenicity of the recombinant proteins was used, namely, the ELISA immunoenzymatic testing.

The immunoenzymatic testing was performed using commercial kits. In the present concretization for the assessment of the astrovirus VP90 recombinant protein, a kit IDEA (DAKO, Inc) was used. Said protein was recognized as shown in FIG. 17.

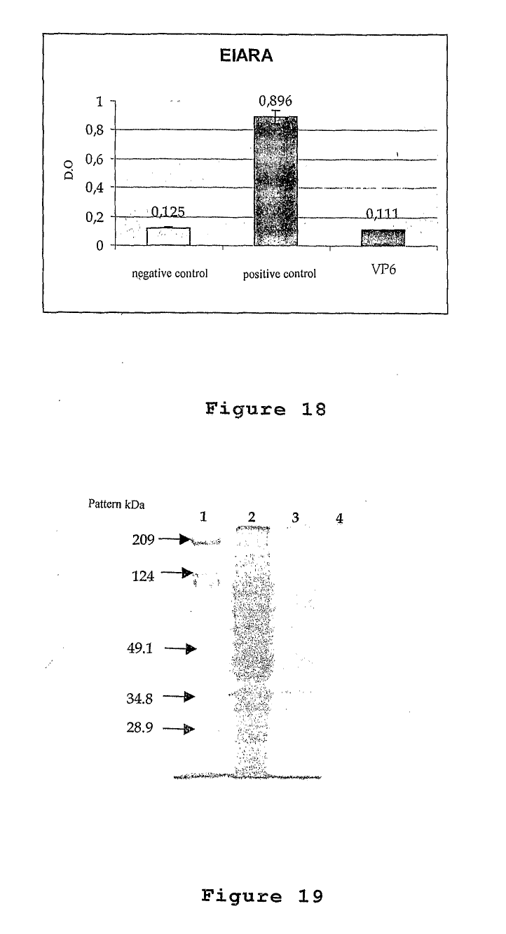

For the assessment of the rotavirus VP90 recombinant protein, an EIARA kit was used. Innumerable tests were performed for this rotavirus VP90 recombinant protein with different concentrations of proteins, but the protein was not recognized by the EIARA kit as shown in FIG. 18.

### Example 12

### Obtainment of the Inclusion Bodies

Approximately 100 mL of induced bacterial culture was used to obtain the inclusion bodies. This induced bacterial culture was centrifuged at 6.500×g for approximately 20 minutes at a preferential temperature of 4° C. to obtain the cellular precipitate. This cellular precipitate was re-suspended in a buffer solution Tris-HCl 20 mM pH 8.0 and then tested by ultrasound under an ice sonication process as described in example 9. The product resulting from the sonication was then centrifuged at 7.000×g for approximately 15 minutes.

After the centrifugation of the sonicated product, the supernatant obtained was discarded and the precipitate was washed at least twice with an appropriate buffer solution, for example, the Tris-HCl pH 8.0 solution, which contained 4 m urea, 0.5M NaCl and 2% X-100 triton so as to re-suspend the precipitate.

After the precipitate re-suspension, the insoluble portion was tested by ultrasound under an ice sonification process as described in example 9. The product resulting from the sonification was centrifuged at 7.000×g for approximately 15 minutes so as to purify the inclusion bodies.

The purified inclusion bodies were re-suspended in an adequate buffer solution, for example, a Tris-HCl pH 8.0 solution containing 6M guanidine hydrochloride, 0.5 NaCl and 5 mM imidazole under constant agitation at a temperature close to 23-26° C. for approximately 1 hour.

After this agitation period, a new centrifugation was performed at 7.000×g for approximately 20 minutes at a preferential temperature of 4° C., so as to separate the non-solubilized portion. The supernatant obtained with this centrifugation was filtered, preferably in a 0.45 μm filter and stored at a preferential temperature of 4° C.

The inclusion bodies thus obtained were analyzed using the electrophoresis technique in SDS-PAGE gel as described in example 8.

FIG. 19 shows the preparation of the inclusion bodies using different buffer solutions in various washing operations, so as to minimize the number of contaminants present during preparation. FIG. 19 shows 1—the pre-stained standard molecular weight (Bio-Rad); 2—Washing with Tris-HCl 20 mM pH 8.0 buffer; 3—First wash with the Tris-HCl pH 8.0 buffer containing 4 m urea, 0.5M NaCl and 2% X-100 triton; 4—Second washing with the Tris-HCl pH 8.0 buffer containing 4M urea, 0.5M NaCl and 2% X-100 triton.

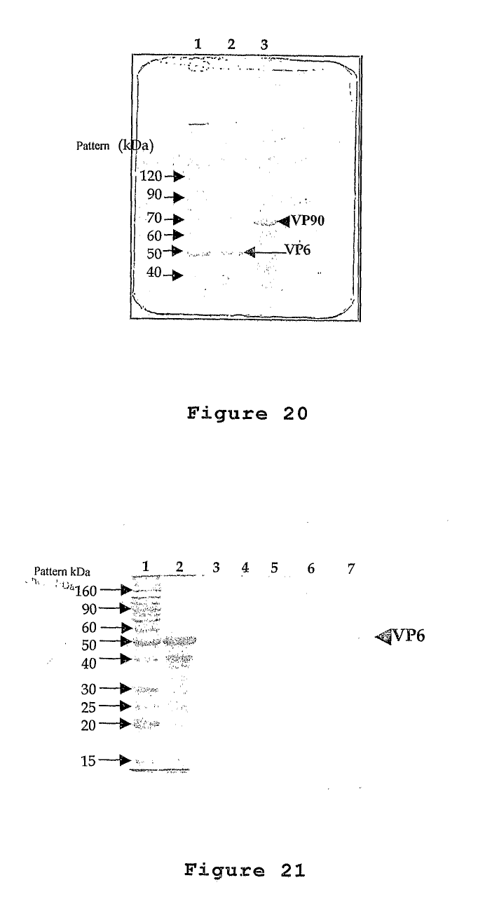

FIG. 20 shows that when the inclusion bodies are purified and solubilized, these inclusion bodies possess a reduced number of contaminants. FIG. 20 shows 1—the standard Molecular weight BenchMark (Invitrogen); 2—Inclusion bodies purified with the BL21(DE) strain containing induced plasmid pOM187 (rotavirus) 3—Inclusion bodies purified with the BL21(DE) strain, containing induced pOM186 plasmid (astrovirus).

### Example 13

### Purification of the Recombinant Proteins Using the Metal Ion Affinity Column

The purification of the recombinant proteins contained in the inclusion bodies solubilized through the metal ion affinity column occurred in such a way that 1.5 mL of the solution containing the purified inclusion bodies was added to a column, which contained approximately 5 mL of a Ni++ Probond resin. This material was incubated for approximately 1 hour at a preferential temperature of 4° C. under constant agitation. At the end of the incubation period, the fluid volume, which contained the proteins that were not able to bind to the resin, was removed. This material, called eluate, was stored at low temperatures for subsequent analysis by the electrophoresis technique in SDS-PAGE gel as described in example 8.

The resin that contained the recombinant proteins was washed with at least twice the volume of the resin used in the purification with a Tris-HCl 0.01M pH 8.0 buffer solution containing 8M urea and 0.1 mM NaH2PO4.

After the washing operation to remove the material not bound to the resin, said resin was again washed so that the contaminants presenting less affinity with the resin could be eliminated. In this second washing operation, a Tris-HCl, 0.01M pH 8.0 buffer solution containing 8M urea, 0.1 mM NaH2PO4 and 10 mM imidazole was used. This second washing operation was performed at least 5 times with approximately 5 mL of the buffer solution with imidazole under constant agitation, for approximately 10 minutes, at a temperature of approximately 4° C.

At the end of the washing operation described above, the resin was washed with the buffer solution Tris-HCl 0.01M pH 8.0, which contained 0.1 mM NaH2PO4 and 10 mM imidazole, so that the urea present in the resin could be removed.

For the elution of the recombinant proteins bound to the nickel resin, approximately 1 mL of a phosphate buffer solution 50 mM pH 4.5 containing 300 mM NaCl and 2M imidazole was used; the column with this buffer was then incubated for approximately 16 hours at a temperature of approximately 4° C. Three to four elutions were performed for each purifying process, all in the same way.

The washed and eluted elute was stored, preferably at 20° C. for subsequent analysis by the electrophoresis technique in SDS-PAGE gel, as described in example 8.

The recombinant proteins, which were eluted during the purification in nickel column, were then put together in an unique sample and dialyzed with a PBS solution 0.2× for approximately 16 hours at a preferably temperature of 4° C. Once the dialysis period ended, the purified recombinant proteins were concentrated approximately 3× in a vacuum system.

FIGS. 21 and 22 show the purification of the recombinants proteins VP6 and VP90, respectively, in which different buffer solutions were used in various washing operations so as to minimize the number of contaminants present in the preparation. FIGS. 21 and 22 show: 1—standard Molecular weight BenchMark (Invitrogen); 2—Purified inclusion bodies; 3—Eluate; 4—Resin washing with a buffer solution Tris-HCl 0.01M pH 8.0 containing 8M urea and 0.1 mM NaH2PO4; 5—Resin washing with a buffer solution Tris-HCl 0.01M pH 8.0 containing 8M urea, 0.1 mM NaH2PO4 and 10 mM imidazol; 6—Resin washing with a buffer solution Tris-HCl 0.01M pH 8.0, containing 0.1 mM NaH2PO4 and 10 mM imidazole; 7—the resin before the protein elution.

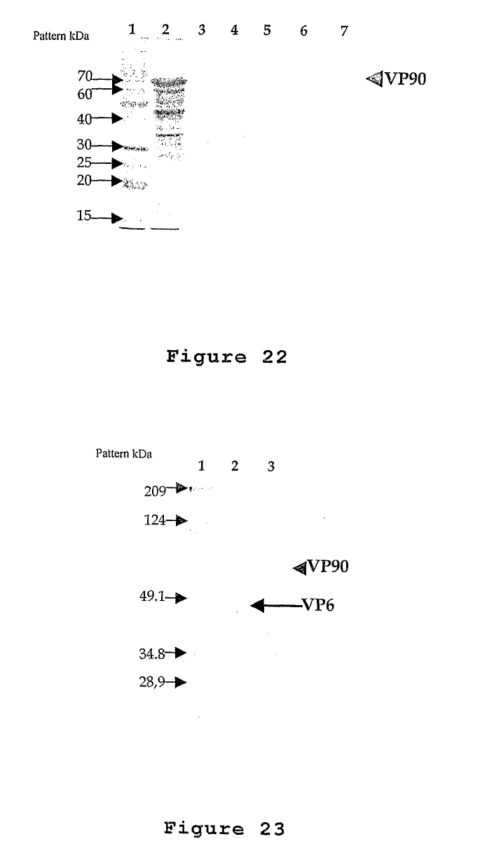

FIG. 23 shows that when the recombinant protein VP6 and VP90 are purified and concentrated, said proteins present a reduced number of contaminants. FIG. 23 shows: 1—the pre-stained standard Molecular weight (Bio-Rad); 2—the rotavirus recombinant protein VP6; 3—the astrovirus recombinant Protein VP90.

### Example 14

### Quantification and Assessment of the Recombinant Protein Purity Level

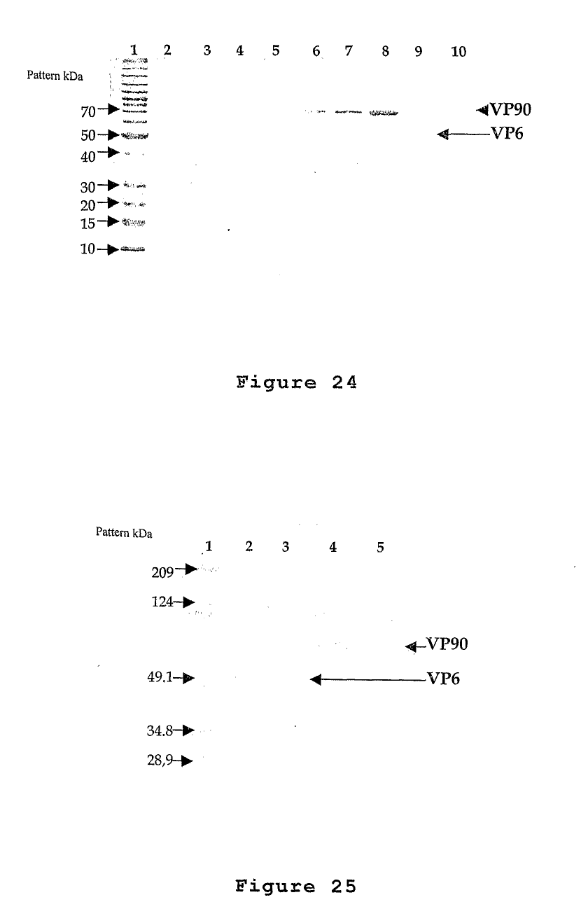

For an estimate of the proteic concentration, an analysis of the concentrate was performed using the electrophoresis technique in SDS-PAGE gel, as described in example 8. For the quantification of the purified and concentrated recombinant proteins, the oliacrilamida gel stained with coomasie-blue was submitted to densitometry, so as to compare the purified and concentrated recombinant proteins with a standard curve of bovine albumin (BSA) as shown in FIG. 24. This Figure shows: 1—the standard Molecular weight BenchMark (Invitrogen); 2 to 8—Standard curve with BSA diluted in concentrations of 5 μg, 10 ug, 20 ug, 30 ug, 40 μg, 50 ug and 100 ug, respectively; 9—rotavirus purified recombinant protein VP6; 10—astrovirus purified recombinant protein VP90.

The estimate of the purity level of the purified recombinant proteins to be inoculated in animals used the electrophoresis technique in SDS-PAGE gel, as described in example 8. In this procedure, a portion of the pool obtained after the purification of said proteins was preferably concentrated at 10× prior to the analysis. FIG. 25 shows the result of the estimate of the purity level of the purified recombinant proteins. This Figure shows that the purified recombinant proteins present a reduced number of contaminants. FIG. 25 shows—1—the pre-stained standard Molecular weight. (Bio-Rad); 2—the ten-time purified and concentrated rotavirus VP6 Protein; 3—the three-time purified and concentrated rotavirus VP6 Protein; 4—the ten-time purified and concentrated rotavirus VP90 Protein; 5—the three-time purified and concentrated rotavirus VP90 Protein.

### Example 15

### Inoculation of the Recombinant Proteins in Animals

For the procedure to inoculate recombinant proteins in animals, four New Zealand rabbits were selected, males, with weight varying from 3 to 3.5 kg.

After the animals' selection, a first bleeding was performed with a 25×7 perforating device without anesthesia. The serum obtained in this bleeding was used as a negative control.

Approximately 15 days after the animals' bleeding, approximately 100 ug of each purified recombinant protein was inoculated in 2 rabbits (100 ug of protein/rabbit) using a perforating device without anesthesia. The inoculation was done on the inner part of the rear paw, intramuscularly.

Four inoculations and a reinforcement inoculation were applied to each rabbit. The recombinant proteins were inoculated in the animals together with a Freud's adjuvant solution. The first recombinant protein inoculation was performed with a complete Freud's adjuvant, whereas the rest of the recombinant protein inoculations were performed using an incomplete Freud's adjuvant.

The interval between the inoculations was of 15 to 20 days.

However, the interval for the reinforcement dose application was preferably 1 month after the last inoculation.

After the end of each inoculation, a partial bleeding was performed with the help of a perforating device, e.g., a scalp size 25 or a needle 25×7, without the use of anesthesia, so to assess the animal's specific immunological response. Approximately 5 mL blood was taken from each animal during the partial bleeding.

A complete bleeding was performed a month after the reinforcement dose inoculation by the cardiac punch technique using a 40×12 perforating device.

During the complete bleeding, the animal remained anesthetized with a hydrochloride ketamine solution of approximately 100 mg/kg live weight.

The polyclonal antibodies analysis was performed using the electrophoresis technique in SDS-PAGE gel as described in example 8. In the electrophoresis, purified recombinant proteins were used as samples, for all the produced antibodies, and in the rotavirus case, the native purified protein VP6 was also used as a sample.

After being submitted to the electrophoresis technique, the proteins were transferred to nitrocellulose membranes by a Transblot system. After the protein transfer to the nitrocellulose membranes, these membranes were blocked with an appropriate blocking solution for approximately 16 hours at a temperature of approximately 4° C. In the present concretization, the blocking solution used was a PBS 1× solution, which contained approximately 0.05 (v/v) Tween 20 and 5% (p/v) Molico milk.

Next, each one of the nitrocellulose membranes was separately incubated with one of the following antibodies: Anti-histidine, which was produced in mice, anti-GST, which was produced in goats and a specific polyclonal antibody (anti-rotavirus produced in goat Chemicon), in addition to the produced polyclonal serums. Said antibodies were previously diluted in a blocking buffer and the membranes were incubated with these solutions containing the antibodies for approximately two hours under soft agitation, at a temperature of approximately 25° C.

The serum obtained after each one of the bleedings, in other words, both the pre and post immunization serums, was tested preferably in a 1/1000 dilution, regardless of the protein immobilized in the membrane. The controls used in this procedure were the specific antibodies for the fusion proteins (histidine and GST) and specific polyclonal antibodies for rotavirus (Chemicon, Inc.), all of them diluted according to the manufacturer's recommendations.

The nitrocellulose membranes were washed at least three times with a PBS 1× solution, which contained approximately 0.05% Tween 20. Each washing operation was performed by incubating the nitrocellulose membranes for approximately 10 minutes with said washing solution. At the end of these three nitrocellulose membranes washing operations, an anti-mouse antibody, an anti-goat antibody or an anti-rabbit antibody was added to said membranes. All of the added antibodies were added together with an alkaline phosphatase and diluted in a blocking buffer solution according to the manufacturer's recommendations. The membranes were incubated with mentioned solutions containing the antibodies for at least 1 hour at a temperature of approximately 23-25° C., under soft agitation. After the incubation period, the membranes were again washed at least three times with the above described PBS 1× solution and incubated as described above.

After washing with the PBS 1× solution, the nitrocellulose membranes were washed at least once with a 100 mM Tris-HCl pH9 buffer solution, which contained approximately 150 mM NaCl and 1 mM CaCl2. The revelation of the nitrocellulose membranes was done by addition of substrate NBT/BCIP (Promega), the above mentioned buffer solution, according to the manufacturer's recommendations.

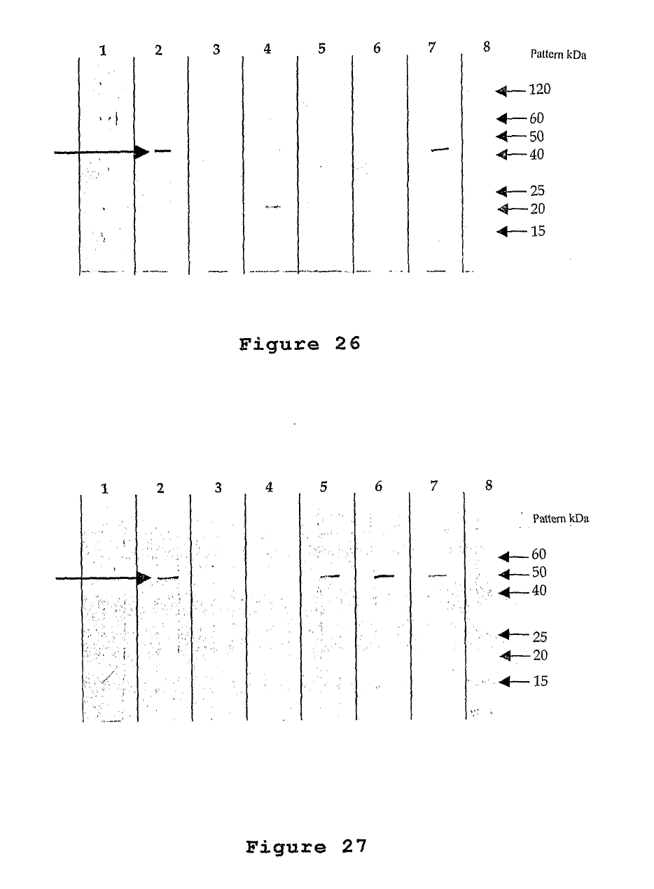

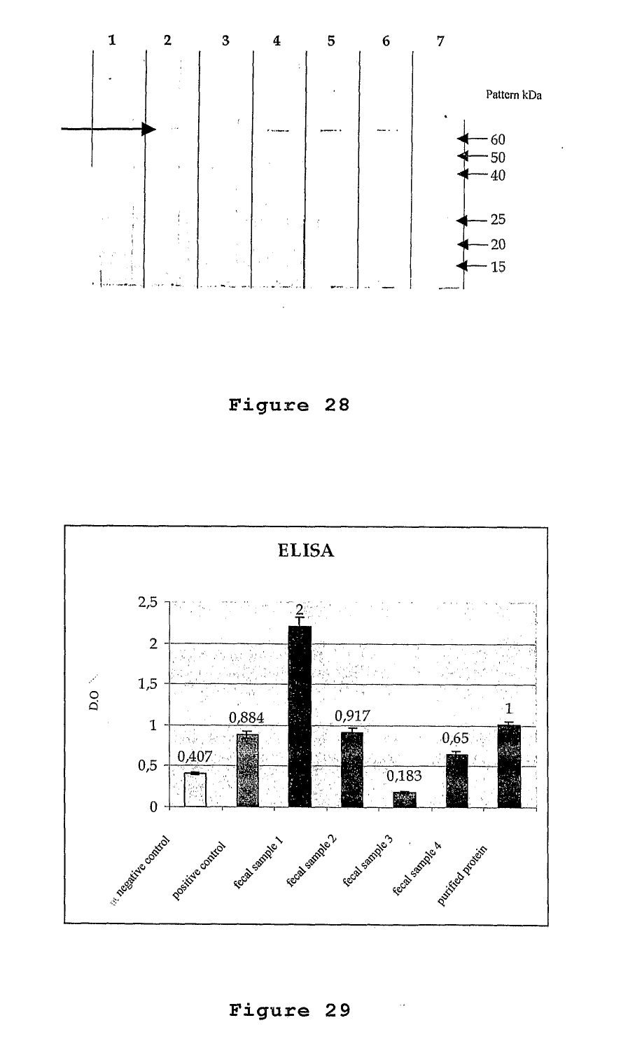

FIGS. 26, 27 and 28 present the results of the immunogenicity evaluation of the recombinant proteins VP6 and VP90. These proteins were able to induce the production of specific immunoglobulins in the immunized rabbits. Said Figures show that the produced polyclonal antibodies were able to recognize the recombinant proteins and in the case of the recombinant anti-VP6, it also recognized the native purified VP6 protein. FIGS. 26 and 27 show: 1—Development with rabbit serum 50 before the recombinant protein inoculation; 2—Development with rabbit serum 50 after the recombinant protein inoculation 3—development with rabbit serum 51 before the recombinant protein inoculation; 4—development with rabbit serum 51 after the recombinant protein inoculation; 5—development with anti-histidine (Invitrogen); 6—Development with anti-GST (GE Health); 7—Development with anti-rotavirus (Chemicon); 8—the pre stained standard Molecular weight BenchMark (invitrogen). FIG. 28 shows; 1—development with rabbit serum 48 before the recombinant protein inoculation; 2—Development with rabbit serum 48 after the recombinant protein inoculation; 3—Development with rabbit serum 24 after recombinant serum inoculation 4—Development with rabbit serum 24 after the recombinant protein inoculation; 5—Development with anti-histidine (invitrogen); 6—Development with anti-GST (GE Health); 7—pre-stained standard Molecular weight BenchMark (Invitrogen).

The serum against rotavirus obtained in partial bleedings was also tested using the ELISA technique. In this test only the post immunization serums with positive results in the Western-blot were used as the capturing antibody in the ELISA test.

The plaque sensitization was done with the serums against recombinant VP6, preferably in the dilution of 1/1000 times in a sodium carbonate 0.016M/sodium bicabornate 0.034M pH 9.6 buffer solution for approximately 16 hours, at a temperature of approximately 4° C.

After this incubation period, the plaques were washed with a buffer washing solution, for example, a PBS 1× solution containing approximately 0.05% Tween 20 and incubated with the samples to be tested for approximately 90 minutes at a preferable temperature of 37° C.

The samples tested during the ELISA testing were feces suspensions at 10° positive for rotavirus, the EIARA kit positive control, the purified and concentrated recombinant protein and the purified SA11 virus from supernatant cell culture. As negative control, a feces suspension at 10° positive for adenovirus, in addition to a conjugated control, was used. All tested samples were previously diluted, preferably ¼ times, in PBS 1× solution, which contained 0.05% Tween 20, 1% bovine albumin (BSA) and 10 mM EDTA (sample diluent). For the combined control, the sample was replaced by the same volume of this same buffer.

The fecal suspension at 10% (v/v) of each of the feces samples was prepared in a buffer solution Tris-HCl 0.01M pH 7.2 containing 0.0015M CaCl2, homogenized in suitable equipment, for example, a vortex, and clarified through the centrifugation technique at 3000×g, for approximately 10 minutes, at a preferable temperature of 4° C.

After the incubation of the capturing serum with the samples, the plaques were washed at least once with a buffer washing solution and submitted to a reaction with specific anti-rotavirus antibody (Chemicon, Inc) preferably diluted 1/1000 in a PBS 1× solution, which contained 0.05 Tween 20 and 1% albumin, for approximately 90 minutes at a preferable temperature of 37° C.

After the incubation, the plaques were washed at least once with the washing buffer solution and again submitted to a reaction with anti-goat antibody conjugated to a peroxidases, preferably diluted 1/1000 in PBS 1× containing 0.05% Tween 20 and 1% albumin, for approximately 90 minutes, at a preferable temperature of 37° C. After the end of this incubation, the plaques were again washed at least once with the washing buffer solution and submitted to the revelation procedure.

The reaction revelation procedure was performed preferably with 100 ul of a peroxide water solution 30 volumes and a concentration of 0.1% (substrate) and 100 ul of a cromogen solution, for example, the Tetrametilbenzidina (TMB-cromogen) at a concentration of 0.01%. Both substances were diluted in a buffer solution of 0.024 3M citrate-0.0514M phosphate pH 5.6. After dilution, the reactions were incubated for approximately 15 minutes in a dark chamber. After the incubation period, the reactions were interrupted with approximately 25 ul of a sulfuric acid solution 2M.

FIG. 29 shows the analysis result using ELISA of the anti-VP6 recombinant polyclonal antibodies. This Figure shows that this antibody was able to detect the rotavirus presence in some feces samples.

The reactions were evaluated on a photometric device fitted with a 450 nm filter. The reaction cut value was calculated based on the D.O. average of the combined control multiplied by two. The samples that presented D.O higher than the cut value obtained were considered positive.

### Example 17

### Immunoglobulin Purification

Immunoglobulins from post-immunization serums were purified and subsequently bound to latex. For this purification, the Bio-Rad Affi-Prep Protein A Matrix kit was used. The purification was performed according to the manufacturer's recommendations.

The collected fractions were evaluated by both spectrophotometer reading at 280 nm and SDS-PAGE electrophoresis according to example 8. Fractions containing purified immunoglobulins were quantified by using the Sigma Bicinchonic Acid Protein Test Kit.

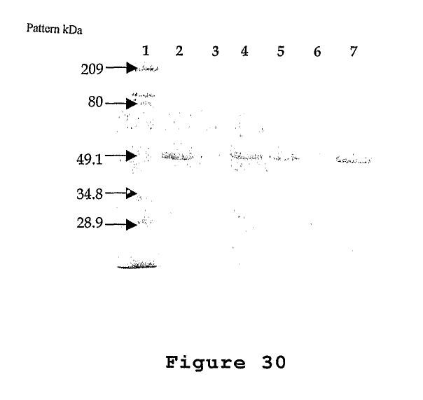

FIG. 30 shows the result of the immunoglobulin purification. As can be observed in FIG. 30, the purified immunoglobulins show two bands. One band represents the light chain and the other one represents the heavy chain. In this figure, 1 is the Bio-Rad pre-stained molecular weight standard and lines 2 to 7 are the purified immunoglobulins.

Therefore, as demonstrated above, the expression, purification and characterization of the rotavirus VP6 protein and astrovirus VP90 protein epitopes, both obtained by the E. coli recombinant protein expression system, were performed. In addition, the use of these recombinant proteins for the combined diagnosis of viral diseases caused by these viruses was also demonstrated.

This invention and its aspects discussed herein should be considered as one of the possible concretizations. However, it should be noted that the invention is not limited to these concretizations. Those people that are familiarized with the technique will realize that the addition of any particular feature should only be understood as a tool to facilitate comprehension and that said additions can only be made by deviating from the described inventive concept. The limiting characteristics of the present invention are related to the claims described hereinafter.

1. Process for the production of immunobiologics for use in a diagnostic kit for gastroenteric viral diseases, wherein the process comprises:
the development of plasmid vectors for protein expression in a E. coli system by means of molecular biology;
the evaluation of the protein profile and yield obtained in a low-scale bacterial culture of clones transformed with plasmid expression vectors;
the evaluation of the antigenicity of the expressed epitopes;
the standardization of a method of purification of the recombinant epitopes of both the rotavirus VP6 protein and astrovirus VP90 protein to be used as immunogens in animal models for the production of polyclonal antibodies;
the characterization of the anti-rotavirus VP6 and anti-astrovirus VP90 polyclonal antibodies derived from specific recombinant proteins.

2. Oligonucleotides containing the sequences SEQ ID. NO: 1, SEQ ID. NO: 2, SEQ ID. NO: 3 and SEQ ID. NO: 4 as described below:

| | |
|---|---|
| CTTCgCCATggAggTTCTgTACTCAC | SEQ ID NO: 1 |
| GTCgCgCCATCggCCgAATTAATTACTC | SEQ ID NO: 2 |
| AATCACTCCATgggAAgCTCCTATgC | SEQ ID NO: 3 |
| GTgACAAgCTCggCCgCAgATACAgC | SEQ ID NO: 4 |

3. Expression plasmidial vectors containing specific regions for expression of recombinant proteins or epitopes of the rotavirus VP6 protein and astrovirus VP90 protein.

4. Expression plasmid vectors, according to claim 3, wherein the cloned sequences correspond to the target regions of the invention: nucleotide position 23 to 742 for VP6 and nucleotide position 4145 to 5325 for VP90.

5. Diagnostic kit for gastroenteric viral disease diagnosis using antibodies derived from recombinant proteins.
